(19) United States
(12) Patent Application Publication
(10) Pub. No.: US 2026/0237709 A1
(43) Pub. Date: Aug. 13, 2026

(54) QUINOXALINE DERIVATIVES FOR AQUEOUS REDOX FLOW BATTERIES

(71) Applicant: REGENTS OF THE UNIVERSITY OF MICHIGAN, Ann Arbor, MI (US)

(72) Inventors: David Gator Kwabi, Milford, CT (US); Bryan R. Goldsmith, Ann Arbor, MI (US); Sanat Vibhas Modak, Waltham, MA (US); Daniel Pert, Pittsburgh, PA (US)

(21) Appl. No.: 19/052,233

(22) Filed: Feb. 12, 2025

(51) Int. Cl. H01M 8/18; C07D 241/40

(52) U.S. Cl. CPC H01M 8/188; C07D 241/40; H01M 2300/0014

(57) ABSTRACT

The disclosure relates to quinoxaline derivatives that are suitable for use as charge carriers in a redox flow battery. A redox flow battery according to the disclosure includes an electrochemical cell containing an electrolyte including a quinoxaline derivative according to Formula I:

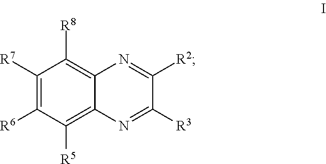

In Formula I, R², R³, R⁵, R⁶, R⁷, and R⁸ are each independently selected from the group consisting of H, a hydrocarbon group containing 1 to 10 carbon atoms, a nitro group, a cyano group, a carbonyl group, a sulfonate group, a sulfonyl group, a hydroxyl group, an ether group, an amino group, a thio group, a phosphino group, and any of the foregoing linked to the quinoxaline ring via a hydrocarbon linking group containing 1 to 10 carbon atoms.

### STATEMENT OF GOVERNMENT SUPPORT

This invention was made with government support under the award number 2033969 granted by the National Science Foundation. The government has certain rights in the invention.

### BACKGROUND OF THE DISCLOSURE

### Field of the Disclosure

The disclosure relates to a redox flow battery including a water-soluble charge carrier compound which is a quinoxaline derivative that is tautomerization-resistant, for example including at least one strong electron withdrawing group at specified positions.

### Brief Description of Related Technology

Renewable energy conversion systems, including solar and wind power installations, have become more common over the last decade. However, they remain limited by the intermittent availability of solar and wind power. Low-cost energy storage technologies are needed to fill the gap between energy supply and demand. Amongst the available energy storage technologies, redox flow batteries (RFBs) are considered promising due to their cost and scalability. RFBs store energy in redox-active charge carriers dissolved in a pair of electrolytes, termed the anolyte (negative electrolyte) and catholyte (positive electrolyte). The electrolytes are stored in separate reservoirs and pumped through an electrochemical cell, where redox reactions store or release electrical energy. This architecture decouples the energy storage and power capacities of the RFB, with the energy storage capacity scaling with the volume and concentration of the charge carriers in their respective electrolytes, and the power scaling with the cell's conductance and electrode area. An RFB's cost ($/KW·h) approaches the cost of its electrolytes as its discharge duration (ratio of energy to power) increases, i.e., as electrolyte's energy storage capacity increases for a fixed power capacity. RFBs therefore may provide cheaper energy storage compared to solid-state batteries if sufficiently inexpensive charge carriers are developed.

Several classes of aqueous-soluble organic molecules have been investigated as potential charge carriers in RFBs, however, most of these molecules undergo rapid decomposition resulting in capacity loss rates during cycling of 0.1%/day or higher.

Substituted anthraquinones and phenazines have been investigated as anolyte molecules; while they have shown acceptable capacity fade rates (ca., ≤0.01%/day) against ferrocyanide (Fe(CN)₆)-based catholytes under alkaline conditions, these molecular pairings have been found to lead to low open-circuit voltages. These low open circuit voltages signify an increase in the cost of energy storage for such anolyte-catholyte pairs, to levels superior to the $150/kW·h target set by the U.S Department of Energy, and too high to be considered for successful commercialization. This highlights the need for further exploration of the organic chemistry design space for more suitable redox-active molecules.

### SUMMARY

In an aspect, the disclosure relates to a quinoxaline derivative according to Formula I:

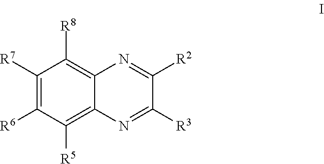

In Formula I, R², R³, R⁵, R⁶, R⁷, and R⁸ are each independently selected from the group consisting of H, a hydrocarbon group containing 1 to 10 carbon atoms (e.g., linear or branched, saturated or unsaturated (such as alkyl, alkenyl, alkynyl), unsubstituted or substituted, such as with one or more N, O, S, P heteroatoms and/or one or more halogen X atoms (e.g., F, Cl, Br, I)), a nitro group (—NO₂), a cyano group (—CN), a carbonyl group (—C(═O)—) (e.g., carboxylate, such as —C(═O)OH (acid) or salt thereof, —C(═O)H (aldehyde), —C(═O)R (ketone, such as with R being a hydrocarbon group containing 1 to 10 carbon atoms), —C(═O)OR (ester, such as with R being a hydrocarbon group containing 1 to 10 carbon atoms), —C(═O)NH₂ (amide, optionally with one or both H atoms substituted with R being a hydrocarbon group containing 1 to 10 carbon atoms), —C(═O)X (acyl)), a sulfonate group (—SO₃H or salt thereof), a sulfonyl group (—SO₂H or —SO₂R, such as with R being a hydrocarbon group containing 1 to 10 carbon atoms), a hydroxyl group (—OH), an ether group (—OR, with R being a hydrocarbon group containing 1 to 10 carbon atoms, or polyether such as a polyalkylene oxide with an ether or hydroxy terminal —(OCₙH₂ₙ)ₘOR or —(OCₙH₂ₙ)ₘOH), an amino group (—NH₂, optionally with one or both H atoms substituted with R being a hydrocarbon group containing 1 to 10 carbon atoms; can include ammonium salts thereof), a thio group (—SH thiol, optionally with the H atom substituted with R being a hydrocarbon group containing 1 to 10 carbon atoms), a phosphino group (—PH₂, optionally with one or both H atoms substituted with R being a hydrocarbon group containing 1 to 10 carbon atoms), and any of the foregoing linked to the quinoxaline ring via a hydrocarbon linking group containing 1 to 10 carbon atoms. Optionally, at least one of conditions (A)-(D) is satisfied for the quinoxaline derivatives: (A) the quinoxaline derivative according to Formula I has a separation between the anodic and cathodic redox potential lower than 0.5 V as measured by cyclic voltammetry at a scan rate of 50 mV/s in an alkaline medium; (B) the quinoxaline derivative according to Formula I has a fade rate constant of 1×10⁻⁶/s or less; (C) the quinoxaline derivative according to Formula I has a tautomerization free energy (ΔGₜₐᵤₜ) greater than 0 kcal/mol; and (D) the quinoxaline derivative according to Formula I has a redox potential in a range of −0.6 V to −1.8 V.

These and other conditions described herein can represent properties, whether individually or collectively, of quinoxaline derivatives that can correspond to compounds particularly suitable for use in a redox flow battery, for example compounds with high stability and long lifetime during use (e.g., tautomerization-resistant compounds with low capacity fade), compounds with good redox properties for efficient charge/discharge of the battery, etc. As described and illustrated below, specific compounds having a selected chemical structure and one or more of these favorable properties can be identified and/or modeled using Density Functional Theory (DFT) as generally known in the art and in the specific context of quinoxaline derivatives, for example to determine tautomerization energetics (e.g., ΔGₜₐᵤₜ) and/or a redox potential (e.g., standard redox potential at pH 13) of a specific quinoxaline derivative.

In another aspect, the disclosure relates to a redox flow battery comprising: an electrochemical cell comprising an electrolyte comprising a quinoxaline derivative (e.g., as a charge-carrier, negative electrolyte molecule/mediator) according to Formula I above; wherein at least one of conditions (A) and (B) is satisfied: (A) R² is selected from the group consisting of a carbonyl group, a cyano group, or a nitro group; and (B) R⁵ and R⁸ are each independently selected from the group consisting of a carbonyl group, a cyano group, or a nitro group.

In another aspect, the disclosure relates to a redox flow battery comprising: an electrochemical cell comprising an electrolyte comprising a substituted quinoxaline, wherein the substituted quinoxaline comprises at least one of (A) a carbonyl group, a cyano group, or a nitro group at position 2, and (B) a carbonyl group, a cyano group, or a nitro group at positions 5 and 8 (e.g., same or different groups at the two positions). More generally, the substituted quinoxaline can include an electron-withdrawing group or other group from R², R⁵, and R⁸ described above at any of positions 2, 5, and/or 8.

In another aspect, the disclosure relates to a redox flow battery comprising: an electrochemical cell comprising an electrolyte comprising a quinoxaline derivative (e.g., as a charge-carrier, negative electrolyte molecule/mediator) according to Formula I above; wherein at least one of conditions (A)-(E) is satisfied: (A) the quinoxaline derivative according to Formula I has a separation between the anodic and cathodic redox potential lower than 0.5 V as measured by cyclic voltammetry at a scan rate of 50 mV/s in an alkaline medium; (B) the quinoxaline derivative according to Formula I has a fade rate constant of 1×10⁻⁶/s or less; (C) the redox flow battery (or electrochemical cell thereof) exhibits a molecular loss rate of 0.1%/day or less; (D) the quinoxaline derivative according to Formula I has a tautomerization free energy (ΔGₜₐᵤₜ) greater than 0 kcal/mol; and (E) the quinoxaline derivative according to Formula I has a redox potential in a range of −0.6 V to −1.8 V. In some embodiments, all or any subset of conditions (A), (B), (C), (D), and (E) are satisfied.

In another aspect, the disclosure relates to an electrolyte solution (e.g., for use in a redox flow battery) comprising: an aqueous medium; and a quinoxaline derivative according to Formula I in solution in the aqueous medium. In embodiments, the quinoxaline derivative is present in a concentration of at least about 0.01 M, or about 0.1 M, between about 0.1 M to about 1.5 M or between about 0.1 M to about 4 M.

In another aspect, the disclosure relates to an electrical power system comprising: a redox flow battery according to the disclosure; and at least one of an electrical power source (e.g., wind, solar, or other (renewable) energy source to charge/recharge the battery, whether directly or indirectly via an electrical power grid) and an electrical load (e.g., any machine or other apparatus electrically driven by the battery, electrical power grid, etc.) in electrical connection with the redox flow battery.

Various refinements of the disclosed compounds, compositions, and apparatus are possible.

In a refinement of Formula I, R² is a carbonyl group (e.g., generically encompassing 2QUIC, 2CHOQUI, 2COCH3QUI). In a further refinement, R² is selected from the group consisting of —C(═O)OH (2QUIC), —C(═O)H (2CHOQUI), and —C(═O)CH₃ (2COCH3QUI); and R³, R⁵, R⁶, R⁷, and R⁸ are each H.

In a refinement of Formula I, R⁵ and R⁸ are each carbonyl groups (e.g., generically encompassing 5,8 QUIDC). In a further refinement, R⁵ and R⁸ are each —C(═O)H; and R²,R³, R⁶, and R⁷ are each H (5,8 QUIDC).

In a refinement of Formula I, R² is a cyano group (e.g., generically encompassing 2CNQUI, DCNQUI, DCNQUI-6,7-DS). In a further refinement, (a) R² is —CN; and R³, R⁵, R⁶, R⁷, and R⁸ are each H (2CNQUI); (b) R² and R³ are each —CN; and R⁵, R⁶, R⁷, and R⁸ are each H (DCNQUI); or (c) R² and R³ are each —CN; R⁵ and R⁸ are each H; and R⁶ and R⁷ are each —SO₃H (DCNQUI-6,7-DS).

In a refinement of Formula I, R² is a nitro group (e.g., generically encompassing 2NO2QUI). In a further refinement, R² is —NO₂; and R³, R⁵, R⁶, R⁷, and R⁸ are each H. (2NO2QUI).

In a refinement, the quinoxaline derivative according to Formula I has a separation between the anodic and cathodic redox potential lower than 0.5 V as measured by cyclic voltammetry at a scan rate of 50 mV/s in an alkaline medium.

In a refinement, the quinoxaline derivative according to Formula I has a fade rate constant of about 1×10⁻⁶/s or less.

In a refinement, the redox flow battery (or electrochemical cell thereof) exhibits a molecular loss rate of 0.1%/day or less (e.g., as measured over 24, 48, or 72 hours of constant current constant voltage (CCCV) cycling).

In a refinement, the quinoxaline derivative according to Formula I has a tautomerization free energy (ΔGₜₐᵤₜ) greater than 0 kcal/mol (e.g., at least 1, 2, 3, 5, 7, 10, 15, or 20 kcal/mol and/or up to 5, 10, 15, 20, 25, 30, 35, 40, or 50 kcal/mol).

In a refinement, the quinoxaline derivative according to Formula I has a redox potential in a range of −0.6 V to −1.8 V (e.g., vs. Ag/AgCl at pH 13).

In a refinement, the quinoxaline derivative according to Formula I has an equivalent weight of 300 g/mol e stored or less (e.g., at least and/or up to 70, 80, 90, 100, 120, 140, 160, 180, 200, 250, or 300 and ranges therebetween).

In a refinement, the quinoxaline derivative according to Formula I has a solubility of at least 0.05 M (e.g., at least and/or up to 0.01, 0.02, 0.05, 0.1, 0.2, 0.4, 1, 1.5, 2.5, or 4 M and ranges therebetween). The solubility can be expressed in the particular electrolyte for a redox flow battery, or it can be expressed in reference (aqueous) solution, for example a 1 M KOH alkaline solution, or an aqueous solution having a pH of 0, 1, 2, 3, 4, 5, 6, 7, 8, 9, 10, 11, 12, 13, 14, or 14.5.

In a refinement, the redox flow battery can further comprise: an anolyte reservoir in fluid communication with the electrochemical cell; a catholyte reservoir in fluid communication with the electrochemical cell; a separator (e.g., ion-exchange membrane) dividing the electrochemical cell into an anolyte compartment and a catholyte compartment; an anode arranged in the anolyte compartment; and a cathode arranged in the catholyte compartment; wherein: the anolyte reservoir is in fluid communication with the anolyte compartment to circulate anolyte solution through the anolyte compartment and the anolyte reservoir; the catholyte reservoir is in fluid communication with the catholyte compartment to circulate catholyte solution through the catholyte compartment and the catholyte reservoir; and the electrolyte comprising the quinoxaline derivative according to Formula I is present in at least one of the anolyte solution and the catholyte solution (e.g., where the quinoxaline derivative can be present in one or both of the anolyte and catholyte solutions depending on whether the cell is an asymmetric or symmetric cell).

In a refinement, the electrolyte (e.g., in the redox flow battery) further comprises an aqueous medium with the quinoxaline derivative according to Formula I in solution in the aqueous medium. Typical concentrations will range between 0.1 M to 4 M. The pH values of the electrolytes can broadly span pH 0-14.5 (e.g., pH of 0, 1, 2, 3, 4, 5, 6, 7, 8, 9, 10, 11, 12, 13, 14, 14.5, and ranges therebetween) during operation. Examples of suitable positive electrolytes or charge carriers can include ferrocyanide (for neutral to alkaline conditions), permanganate (strongly alkaline conditions), ferrocene derivatives (neutral pH), bromide and iodide (acidic conditions).

### BRIEF DESCRIPTION OF THE DRAWINGS

FIG. 1 shows side by side the cyclic voltammograms of a 1 mM DMeQUIC solution and a sodium ferrocyanide solution performed at a scan rate of 50 mV/s in an aqueous supporting electrolyte of 0.1 M NaOH and 1 M NaCl (pH 13).

FIG. 2A is a plot of the Potential (E) vs capacity of DMeQUIC for shallow discharge (1.0 V discharge limit) and deep discharge (−0.1 V discharge limit) cycling measured according to the disclosure.

FIG. 2B is a graph of the charge and discharge capacities vs time during shallow- and deep-discharge CCCV cycling at 20 mA/cm².

FIGS. 3A-3C show the operando UV-vis spectra of a 5 mM DMeQUIC solution in a DMeQUIC-Fe(CN)₆ flow cell (A) before cycling, (B) after charging the cell (reducing DMeQUIC) at 1.6 V and (C) discharging the cell at −0.2 or 1.0 V.

FIG. 3D illustrates the redox reaction that converts DMeQUIC to rDMQ and subsequent tautomerization of rDMQ into redox-inactive tautomers fDMQ. Tautomerization free energies calculated by DFT at pH 13, 1 M species, and 25° C. are shown.

FIG. 3E illustrates the structures and reactions converting the same for a quinoxaline derivative in oxidized form (DMe-QUIC), reduced enamine form (rDMQ), deprotonated enamine (dDMQa and dDMQb), deprotonated imine (tDMQa and tDMQb), and reduced imine form (fDMQa and fDMQb).

FIG. 4A shows the structures of the molecules studied by DFT modeling and cycled experimentally.

FIG. 4B shows the structures of the molecules that were only evaluated by DFT modeling.

FIG. 4C is a plot of the DFT calculated Tautomerization Free energy versus the DFT-computed redox potential at pH 13, 1 M species, and 298 K. Data points of molecules studied experimentally are indicated by squares and other molecules evaluated only by DFT by circles.

FIG. 4D is a plot of the calculated Tautomerization Free energy versus the Hammett constant of the substituent in the position adjacent to a pyrazine N for quinoxaline derivatives functionalized only in the position adjacent to the pyrazine N.

FIG. 5 is a graph of the charge capacity, discharge capacity, and Coulombic efficiency versus time during cycling of a compositionally symmetric 2QUIC cell. The capacity-limiting side initially comprised a half-reduced (50% SOC) solution of 7.5 mL of 0.05 M 2QUIC and 1 M NaOH; the non-capacity-limiting side comprised 18 mL of the same composition.

FIG. 6 is a plot of the charge capacity, discharge capacity, and Coulombic efficiency versus time of selected cycles for the cycling of a mixed symmetric 2QUIC-ferrocyanide cell for 220 h. The capacity-limiting side of the cell comprised 3 mL of 0.1 M 2QUIC, 0.2 M Na₄Fe(CN)₆, and 0.2 M NaOH; the non-capacity-limiting side comprised 7 mL of the same solution.

FIGS. 7A-7B are UV-vis spectra of cycled and uncycled DMeQUIC electrolytes from the cells cycled with a discharge potential limit of (A) 1.0 V and (B) −0.1 V. The electrolytes were diluted by a factor of 4000 with a 3 M NaOH supporting electrolyte to reduce the nominal DMeQUIC concentration from 400 mM to 0.1 mM.

FIGS. 7C-7D are cyclic voltammograms of uncycled and cycled DMeQUIC and electrolytes from cells cycled with a discharge potential limit of (C) 1.0 V and (D)-0.1 V. The electrolytes were diluted by a factor of 400 in 3 M NaOH to arrive at a nominal DMeQUIC concentration of 1 mM and the scans were taken at 50 mV/s. The flow cells had a capacity-limiting electrolyte comprising 6 mL of 0.4 M DMeQUIC and 0.5 M NaOH and a non-capacity-limiting electrolyte comprising 20 mL of 0.4 M Na4Fe(CN)6 and 0.1 M NaOH.

FIGS. 8A-8B are LC-MS scans of (A) uncycled DMeQUIC and (B) DMeQUIC after reduction in a flow cell and storage for seven days in nitrogen atmosphere.

FIG. 9 is a graph of the DFT-calculated free energies of tautomerization, and Michael addition sorted by increasing tautomerization energy for (left panel) five quinoxaline derivatives experimentally tested and (right panel) eight additional quinoxaline derivatives not experimentally tested. When there are two possible tautomers, the more exothermic tautomerization energy is reported.

FIG. 10 is a schematic of the mechanism for Michael attack of the reduced imine form of DMeQUIC (fDMQa/b) to form hydroxylated intermediate (hDMQa/b) followed by Michael adduct (mDMQa/b).

FIG. 11 is a plot of the cathodic peak current decrease percent versus the cell cycling capacity loss percent estimated from the decrease in cathodic redox peak currents in compared to the capacity loss observed during cell cycling.

FIG. 12 is a pair of cyclic voltammograms of uncycled and cycled electrolytes from the capacity-limiting side of the 2QUIC mixed symmetric cell conducted at a 50 mV/s scan rate in a 3 M NaOH supporting electrolyte.

FIG. 13 illustrates a redox flow battery according to the disclosure.

### DETAILED DESCRIPTION

Quinoxalines are an underexplored but promising class of negative electrolyte molecules because many derivatives have lower redox potentials and higher solubilities than typical anthraquinones and phenazines, but their use has been hindered by their known stability issues. The equivalent weight of quinoxaline (65 g/mol e) is lower than that of anthraquinone (104 g/mol e) and phenazine (90 g/mol e), and many substituted quinoxalines are straightforward to synthesize in one-step reactions using widely available precursors. Like anthraquinones and phenazines, quinoxalines undergo a two-electron redox reaction. Although they are known to be viable charge carriers in nonaqueous flow batteries, studies on the cycling behavior of aqueous flow cells containing quinoxalines have reported rapid capacity fade, equivalent to >20%/day. Progress in developing stable aqueous-soluble quinoxalines has been hindered by the poorly understood connection between capacity fade and molecular decomposition mechanisms. For example, although reduced quinoxalines are known to undergo tautomerization, their impact on capacity retention in flow cells is unclear. Other studies have suggested quinoxaline dimerization as the primary degradation mechanism driving capacity fade in alkaline electrolytes, while other reports have proposed Michael attack or irreversible hydrogenation as possible decomposition mechanisms.

The disclosure relates to understanding the mechanisms of decomposition of quinoxalines, the substituent impact on quinoxaline stability, capacity fade, and selection of quinoxaline derivatives for RFB systems possessing long lifetimes and thus the ability to deliver electricity at low costs.

The foregoing description is given for clearness of understanding only, and no unnecessary limitations should be understood therefrom, as modifications within the scope of the disclosure may be apparent to those having ordinary skill in the art.

All patents, publications and references cited herein are hereby fully incorporated by reference. In case of conflict between the present disclosure and incorporated patents, publications and references, the present disclosure should control.

### Definitions

The term quinoxaline refers to a heterocyclic compound containing a ring complex made up of a benzene ring and a pyrazine ring such as

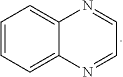

The term “alkyl” can refer to a saturated straight hydrocarbon chain or saturated branched chain hydrocarbon containing the indicated number of carbon atoms. Non-limiting examples of alkyl groups include methyl, ethyl, n-propyl, isopropyl, n-butyl, sec-butyl, isobutyl, tert-butyl, n-pentyl, and n-hexyl.

The term “alkenyl” can refer to a straight or branched hydrocarbon chain containing the indicated number of carbon atoms and having one or more carbon-carbon double bonds. Non-limiting examples of alkenyl groups include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, and 5-hexenyl.

The term “alkynyl” can refer to a straight or branched chain hydrocarbon containing the indicated number of carbon atoms and having one or more carbon-carbon triple bonds. Non-limiting examples of alkynyl groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl.

The term “cyano” can refer to a —CN group.

The term “amino” can refer to a —NH₂ group.

The terms “hydroxy” and “hydroxyl” are interchangeable and can refer to a —OH group.

The term “nitro” can refer to a —NO₂ group.

The term “ether” can refer to a single oxygen atom bonded to two separate carbon atoms, each part of an alkyl group.

The term “heteroatom,” unless otherwise stated herein, can refer to any atom that is not carbon or hydrogen, for example oxygen, sulfur, nitrogen, and/or phosphorus.

The term “halogen,” unless otherwise stated herein, can refer to chlorine, fluorine, bromine, and iodine.

The term “sulfonate” can refer to —S(═O)₂—O.

The term “sulfonyl” can refer to

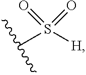

also represented as —SO₂H.

The term “carbonyl” can refer to a divalent C═O radical, such as

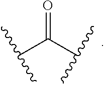 [SMILES: *C(*)=O]

For example, aldehyde groups

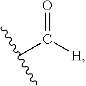

ketone groups

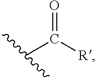

carboxylic acid groups

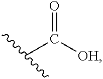 [SMILES: *C(=O)O]

and carboxylate esters

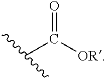

The term “substituent” refers to a functional group replacing one or more hydrogen atoms in a given structure or functional group. A substituent may be located at any substitutable position of the structure or functional group. When more than one position in a given structure can be substituted with more than one substituent, the substituent may be either the same or different at each position.

### Quinoxaline Derivatives of Formula I

A quinoxaline derivative according to the disclosure can be represented by Formula I:

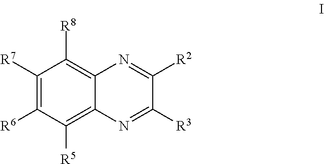

The quinoxaline derivative can be used as a charge-carrier or negative electrolyte molecule/mediator in a redox flow battery (e.g., as a component of an electrolyte solution therein). In Formula I, R², R³, R⁵, R⁶, R⁷, and R⁸ (or “R^{x}” collectively) can be the same or different substituents. Each R^{x} group in Formula I can be a substituent independently selected from H (hydrogen atom), a hydrocarbon group containing 1 to 10 carbon atoms, a nitro group, a cyano group, a carbonyl group, a sulfonate group, a sulfonyl group, a hydroxyl group, an ether group, an amino group, a thio group, and a phosphino group, for example covalently bonded directly to the quinoxaline ring at the various positions indicated above. In embodiments, each R^{x} group in Formula I can include any of the foregoing substituents indirectly bonded to the quinoxaline ring via a hydrocarbon linking group containing 1 to 10 carbon atoms between the substituent and the selected position on the quinoxaline ring (e.g., with the linking group being covalently bonded to both the quinoxaline ring and the R^{x} group). Quinoxaline derivatives according to the disclosure can be formed using conventional chemical synthetic techniques as generally known in the art, for example starting from a quinoxaline substrate or a different quinoxaline derivative substrate. In some embodiments, R² can be selected to be a carbonyl group, a cyano group, or a nitro group. In some embodiments, R⁵ and R⁸ can be independently selected to be a carbonyl group, a cyano group, or a nitro group.

The hydrocarbon group containing 1-10 carbon atoms is not particularly limited and can be, for example, a linear or branched, saturated or unsaturated, unsubstituted or substituted hydrocarbon chain or group. Examples of saturated or unsaturated hydrocarbon groups include alkyl, alkenyl or alkynyl chains or groups. The hydrocarbon group can be unsubstituted (e.g., consisting of only carbon and hydrogen atoms). Alternatively, the hydrocarbon group can be substituted such as with one or more heteroatoms (e.g., N, O, S, and/or P), which can be a pendant and/or backbone atom or group, and/or one or more halogen atoms (e.g., F, Cl, Br, and/or I, such as perhaloalkyl groups or perhalo carbon atoms). In embodiments, the hydrocarbon group can contain 1, 2, 3, 4, 5, 6, 7, 8, 9, or 10 carbon atoms, or any range therebetween.

In embodiments, the hydrocarbon group containing 1-10 carbon atoms described above can be a direct substituent on the quinoxaline ring, for example at R², R³, R⁵, R⁶, R⁷, and/or R⁸. Alternatively or additionally, the hydrocarbon group containing 1-10 carbon atoms can be a component or substituent of a different group selection for R², R³, R⁵, R⁶, R⁷, and/or R⁸. For example, when one of the R^{x} groups in Formula I is selected to be a carbonyl group (—C(═O)—) in the specific form of an ester group (—C(═O)OR), the hydrocarbon group of the ester (R) can include any of the various selections for the hydrocarbon group containing 1-10 carbon atoms described above. Other examples are described below. Alternatively or additionally, the hydrocarbon group containing 1-10 carbon atoms described above can represent a linking group between the substituent and the selected position on the quinoxaline ring. For example, when one of the R^{x} groups in Formula I is selected to be a cyano group (—CN), the cyano group can be directly bonded to the quinoxaline ring or the cyano group can be indirectly bonded to the quinoxaline ring via an intervening linking hydrocarbon group containing 1-10 carbon atoms according to any of the options described above for the hydrocarbon group containing 1-10 carbon atoms. The linking hydrocarbon group is analogous to the corresponding hydrocarbon group, but including two covalent bonding locations instead of one (e.g., a methylene (·CH₂·) linking hydrocarbon group is analogous to a methyl (·CH₃) hydrocarbon group, an ethylene (·CH₂CH₂·) linking hydrocarbon group is analogous to an ethyl (·CH₂CH₃) hydrocarbon group, etc.).

In embodiments, R², R³, R⁵, R⁶, R⁷, and/or R⁸ can be selected to be a nitro group (—NO₂), for example being directly bonded to the quinoxaline ring or indirectly bonded to the quinoxaline ring via an intervening linking hydrocarbon group containing 1-10 carbon atoms.

In embodiments, R², R³, R⁵, R⁶, R⁷, and/or R⁸ can be selected to be a cyano group (—CN), for example being directly bonded to the quinoxaline ring or indirectly bonded to the quinoxaline ring via an intervening linking hydrocarbon group containing 1-10 carbon atoms.

In embodiments, R², R³, R⁵, R⁶, R⁷, and/or R⁸ can be selected to be a carbonyl group (—C(═O)—), for example being directly bonded to the quinoxaline ring or indirectly bonded to the quinoxaline ring via an intervening linking hydrocarbon group containing 1-10 carbon atoms. In more specific embodiments, the carbonyl group can be a carboxylate, such as —C(═O)OH (acid) or salt thereof (e.g., alkali or other metal salt). In more specific embodiments, the carbonyl group can be an aldehyde (—C(═O)H). In more specific embodiments, the carbonyl group can be a ketone (—C(═O)R), such as with R being a hydrocarbon group containing 1 to 10 carbon atoms as described above. In more specific embodiments, the carbonyl group can be an ester (—C(═O)OR), such as with R being a hydrocarbon group containing 1 to 10 carbon atoms as described above. In more specific embodiments, the carbonyl group can be an amide (—C(═O)NH₂), such as with one or both amide H atoms being substituted with R being a hydrocarbon group containing 1 to 10 carbon atoms as described above. In more specific embodiments, the carbonyl group can be an acyl group (—C(═O)X), such as with X being a halogen atom.

In embodiments, R², R³, R⁵, R⁶, R⁷, and/or R⁸ can be selected to be a sulfonate group (—SO₃—), for example being directly bonded to the quinoxaline ring or indirectly bonded to the quinoxaline ring via an intervening linking hydrocarbon group containing 1-10 carbon atoms. In more specific embodiments, the sulfonate group can be a sulfonic acid (—SO₃H) or salt thereof (e.g., alkali or other metal salt). In more specific embodiments, the sulfonate group can be a sulfonic ester (—SO₃R), such as with R being a hydrocarbon group containing 1 to 10 carbon atoms as described above.

In embodiments, R², R³, R⁵, R⁶, R⁷, and/or R⁸ can be selected to be a sulfonyl group (—SO₂—), for example being directly bonded to the quinoxaline ring or indirectly bonded to the quinoxaline ring via an intervening linking hydrocarbon group containing 1-10 carbon atoms. In more specific embodiments, the sulfonyl group can include a hydrogen substituent (—SO₂H) or a hydrocarbon substituent (—SO₂R), such as with R being a hydrocarbon group containing 1 to 10 carbon atoms as described above.

In embodiments, R², R³, R⁵, R⁶, R⁷, and/or R⁸ can be selected to be a hydroxyl group (—OH), for example being directly bonded to the quinoxaline ring or indirectly bonded to the quinoxaline ring via an intervening linking hydrocarbon group containing 1-10 carbon atoms.

In embodiments, R², R³, R⁵, R⁶, R⁷, and/or R⁸ can be selected to be an ether group (e.g., mono-, oligo-, or polyether), for example being directly bonded to the quinoxaline ring or indirectly bonded to the quinoxaline ring via an intervening linking hydrocarbon group containing 1-10 carbon atoms. In more specific embodiments, the ether group can be a monoether (—OR), such as with R being a hydrocarbon group containing 1 to 10 carbon atoms as described above. In more specific embodiments, the ether group can be a mono-, oligo-, or polyether such as a mono-, oligo-, or polyalkylene oxide with an ether terminal (e.g., —(OCₙH₂ₙ)ₘOR) or hydroxy terminal (e.g., —(OCₙH₂ₙ)ₘOH) group. For the alkylene oxide groups, n can be 1, 2 (e.g., ethylene oxide such as in a polyethylene glycol group), 3 (e.g., propylene oxide), 4, 5, 6, 7, or 8, and m can be 1 to 100 (e.g., at least and/or up to 1, 2, 4, 6, 8, 10, 15, 20, 30, 40, 50, 60, 70, 80, 90, or 100 and ranges therebetween). For an ether terminal group, R can be a hydrocarbon group containing 1 to 10 carbon atoms as described above.

In embodiments, R², R³, R⁵, R⁶, R⁷, and/or R⁸ can be selected to be an amino group (e.g., —NH₂), for example being directly bonded to the quinoxaline ring or indirectly bonded to the quinoxaline ring via an intervening linking hydrocarbon group containing 1-10 carbon atoms. In more specific embodiments, one or both amino H atoms can be substituted with R being a hydrocarbon group containing 1 to 10 carbon atoms as described above (e.g., forming a (di)alkylamino group). In more specific embodiments, the amino group can include ammonium salts thereof (e.g., a substituted or unsubstituted ammonium salt —NH₃X with halogen X).

In embodiments, R², R³, R⁵, R⁶, R⁷, and/or R⁸ can be selected to be a thio group (e.g., —S—, or —SH more specifically as a thiol), for example being directly bonded to the quinoxaline ring or indirectly bonded to the quinoxaline ring via an intervening linking hydrocarbon group containing 1-10 carbon atoms. In more specific embodiments, the thiol H atom can be substituted with R being a hydrocarbon group containing 1 to 10 carbon atoms as described above (e.g., forming a thioether group).

In embodiments, R², R³, R⁵, R⁶, R⁷, and/or R⁸ can be selected to be phosphino group (e.g., —PH₂), for example being directly bonded to the quinoxaline ring or indirectly bonded to the quinoxaline ring via an intervening linking hydrocarbon group containing 1-10 carbon atoms. In more specific embodiments, one or both phosphino H atoms can be substituted with R being a hydrocarbon group containing 1 to 10 carbon atoms as described above (e.g., forming a (di)alkylphosphino group).

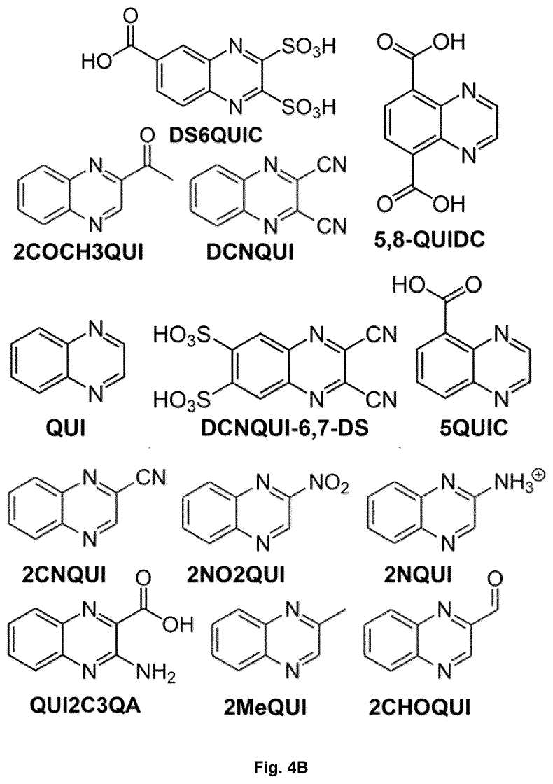

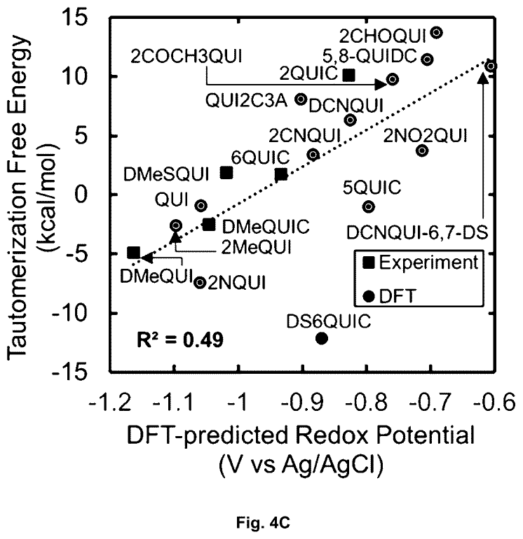

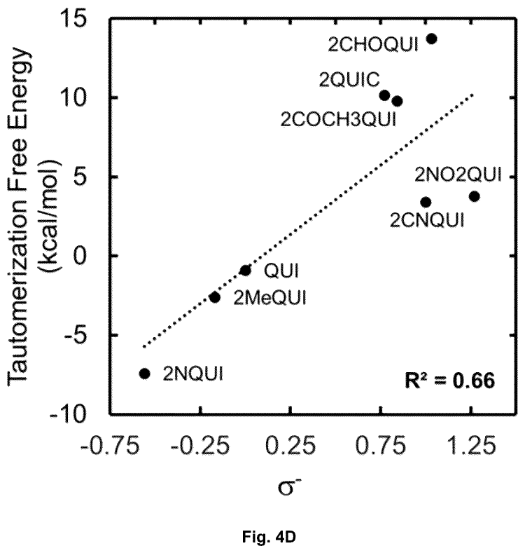

FIGS. 4A and 4B illustrate example quinoxaline derivatives according to Formula I. Specific selections within the general Formula I are described below.

In one embodiment the quinoxaline derivative according to the disclosure can have an R² carbonyl. Furthermore, the quinoxaline derivative can have R³, R⁵, R⁶, R⁷, and R⁸ each to be hydrogen. For example, quinoxaline-2-carboxylic acid (2QUIC)

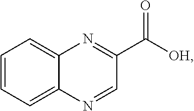 [SMILES: O=C(O)c1cnc2ccccc2n1]

quinoxaline-2-carbaldehyde (2CHOQUI)

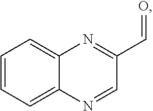 [SMILES: O=Cc1cnc2ccccc2n1]

and 2-acetylquinoxaline (2COCH₃QUI)

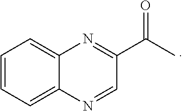

In another embodiment, the quinoxaline derivative can have R⁵ and R⁸ to be carbonyl groups. Furthermore, R⁵ and R⁸ can be carbonyl groups and R², R³, R⁶ and R⁷ can each be hydrogen. For example, R⁵ and R⁸ can be carboxylic acid groups and R², R³, R⁶ and R⁷ can each be hydrogen as in quinoxaline-5,8-dicarboxylic acid (5,8-QUIDC)

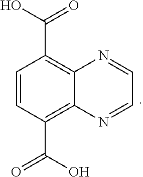 [SMILES: O=C(O)c1ccc(C(=O)O)c2nccnc12]

In another embodiment, the quinoxaline derivative can have an R² cyano group combined with other substituents. For example, the quinoxaline derivative with an R² cyano group and R³, R⁵, R⁶, R⁷ and R⁸ are each hydrogen as in quinoxaline-2-carbonitrile (2CNQUI)

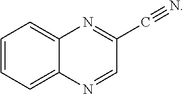 [SMILES: N#Cc1cnc2ccccc2n1]

For example, the quinoxaline derivative with an R² cyano group, a second cyano group at R³, and R⁵, R⁶, R⁷ and R⁸ are each hydrogen as in quinoxaline-2,3-dicarbonitrile (DCNQUI)

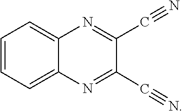 [SMILES: N#Cc1nc2ccccc2nc1C#N]

For example, the quinoxaline derivative with an R² cyano group, a second cyano group at R³, R⁵ and R⁸ are each hydrogen and R⁶ and R⁷ are each sulfonyl groups as in quinoxaline-2,3-dicarbonitrile-6,7-disulfonic acid (DCNQUI-6,7-DS)

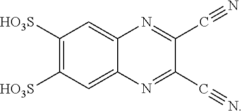 [SMILES: N#Cc1nc2cc(S(=O)(=O)O)c(S(=O)(=O)O)cc2nc1C#N]

In another embodiment, the quinoxaline derivative can have an R² nitro group. For example, the quinoxaline derivative with an R² nitro group can have R³, R⁵, R⁶, R⁷, and R⁸ are each hydrogen as in 2-nitroquinoxaline (2NO2QUI)

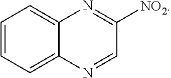 [SMILES: O=[N+]([O-])c1cnc2ccccc2n1]

### Electrochemical Properties and Redox Flow Battery

Quinoxaline derivatives according to the disclosure can have a separation between the anodic and cathodic redox potential lower than about 0.5 V as measured by cyclic voltammetry at a scan rate of 50 mV/s in an alkaline medium. For example, the anodic-cathodic separation redox potential measured by cyclic voltammetry at a 50 mV/s scan rate in alkaline medium can be about 0.5 V, 0.45 V, 0.4 V, 0.35 V, 0.3 V, 0.25 V, 0.2 V, 0.15 V, 0.1 V, 0.05 V, 0.01 V, −0.1 V, −0.2 V, −0.3 V, −0.4 V, −0.5 V, −0.6 V, −0.7 V, −0.8 V, −0.9 V, −1.0 V, −1.1 V, −1.2 V, −1.3 V, −1.5 V or any values therebetween.

Quinoxaline derivatives of the disclosure can be characterized by a fade rate constant of about 1×10⁻⁶/s or less. Capacity fade rate constants can be estimated form cycling the compound in a flow cell (e.g., at about 20-25° C.) and calculating the slope of the linear best fit for normalized discharge capacity on a logarithmic scale against time, for example by cycling the compound over a period of 1, 2, 3, 5, 7, 10, 14, 21, 28, 30, 35, or 42 days or ranges therebetween. For example, estimated capacity fade rate constants can be up to and/or at least about 1×10⁻⁶/s, 0.9×10⁻⁶/s, 0.7×10⁻⁶/s, 0.5× 10⁻⁶/s, 0.3×10⁻⁶/s, 0.2×10⁻⁶/s, 1×10⁻⁷/s, 1×10⁻⁸/s, 1×10⁻⁹/s, 1×10⁻¹⁰/s, or any values therebetween. In some cases, a particular quinoxaline derivative might degrade so slowly that a loss rate is immeasurable at room temperature over about 4-5 weeks of cycling. In such cases, the capacity fade rate constant can be expressed as an upper threshold (e.g., the fade rate constant is not more than a specified threshold level determined based on the length of cycling).

Redox flow batteries including quinoxaline derivatives of the disclosure, can be characterized by a molecular loss rate of about 0.1%/day as measured by cycling the compound in a flow cell (e.g., at about 20-25° C.) over a period of 1, 2, 3, 5, 7, 10, 14, 21, 28, 30, 35, or 42 days or ranges therebetween. For example, the molecular loss rate can be up to and/or at least about 0.1%/day, 0.09%/day, 0.08%/day, 0.07%/day, 0.06%/day, 0.05%/day, 0.04%/day, 0.03%/day, 0.02%/day, 0.01%/day, 0.001%/day, 0.0001%/day, 0.00001%/day, or any values therebetween.

Quinoxalines of the disclosure can be tautomerization resistant, i.e., can be characterized by tautomerization free energies (ΔGₜₐᵤₜ) greater than 0 kcal/mol. As explained and illustrated in the examples below, tautomerization can undesirably convert an otherwise redox-active quinoxaline derivative into a redox-inactive tautomer (e.g., a structural or constitutional isomer or the original quinoxaline derivative structure), thus reducing or eliminating the ability of the quinoxaline derivative to serve as an effective charge carrier in a redox flow battery for extended periods. A positive tautomerization free energy limits or prevents tautomerization, tending to maintain the quinoxaline derivative in its original redox-active form. For example, the free energy of tautomerization can be at least and/or up to about 0.5 kcal/mol, 1 kcal/mol, 2 kcal/mol, 3 kcal/mol, 5 kcal/mol, 10 kcal/mol, 15 kcal/mol, 20 kcal/mol, 25 kcal/mol, 30 kcal/mol, 35 kcal/mol, 40 kcal/mol, 50 kcal/mol, or in a range about 1 kcal/mol to about 50 kcal/mol, about 1 kcal/mol to about 40 kcal/mol, 1 kcal/mol to about 30 kcal/mol, or any values and ranges therebetween.

According to the disclosure, quinoxaline derivatives of Formula I, can have a redox potential measured in alkaline medium (e.g., at pH13) in a range of about −0.6 V to −1.8 V versus a standard Ag/AgCl electrode. For example, alkaline redox potential measured versus the Ag/AgCl electrode can be about −0.6 V, −0.7 V, −0.8 V, −0.9 V, −1.1 V, −1.2V, −1.3V, −1.4V, −1.5V, −1.6V, −1.7V, −1.8V, or any values therebetween.

Redox flow batteries including quinoxaline derivatives of the disclosure, can have an equivalent weight of up to 100, 200, or 300 g/mol e stored. For example, equivalent weight values can be at least and/or up to 70, 80, 90, 100, 120, 140, 160, 180, 200, 250, or 300 g/mol e stored and ranges therebetween.

Quinoxaline derivatives according to the disclosure can be water soluble and soluble in aqueous alkaline solutions in concentrations of at least 0.01 M or 0.05 M, for example in a range of 0.01 M to about 4 M or 0.1 M to 1.5 M. In embodiments, the solubility of the quinoxaline derivative can be at least and/or up to 0.01, 0.02, 0.05, 0.1, 0.2, 0.4, 0.5, 0.6, 0.7, 0.8, 0.9, 1, 1.2, 1.5, 2, 2.5, 3, 3.5, or 4 M and ranges therebetween. The solubility can be expressed in the particular electrolyte for a redox flow battery, or it can be expressed in reference (aqueous) solution, for example a 1 M KOH alkaline solution, or an aqueous solution having a pH of 0, 1, 2, 3, 4, 5, 6, 7, 8, 9, 10, 11, 12, 13, 14, or 14.5. Aqueous alkaline solutions can be for example, NaOH and KOH solutions of concentrations in a range of about 0.1 M to 5 M (e.g., at 0.1, 0.2, 0.5, 1, 2, or 5 M). As illustrated in the examples for the specific case of 2QUIC as the quinoxaline derivative, the solubility in a 1M NaOH aqueous solution can be about 0.45 M, and the solubility in a 1M KOH aqueous solution can be about 1.22 M.

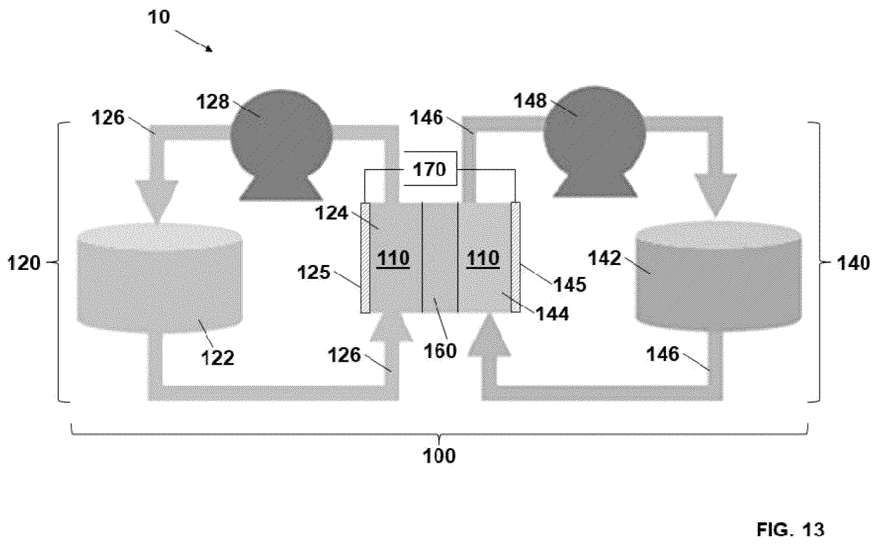

FIG. 13 illustrates a redox flow battery 10 according to the disclosure. The battery 10 can include an electrochemical cell 100 containing an electrolyte 110. The electrolyte 110 can include a quinoxaline derivative charge carrier. The quinoxaline derivative can have a structure according to Formula I described above. In embodiments, the redox flow battery 10 can include an anolyte portion or cell 120 and a catholyte portion or cell 140 separated by a separator 160 such as an ion-exchange membrane as generally known in the art for redox flow batteries. The anolyte portion 120 can include an anolyte reservoir 122 in fluid communication with the electrochemical cell 100, and the catholyte portion 140 can include a catholyte reservoir 142 in fluid communication with the electrochemical cell 100. The separator 160 divided the electrochemical cell 100 into an anolyte compartment 124 and a catholyte compartment 144. Further, the battery 10 can include electrodes 125, 145 such as an anode 125 positioned or arranged in the anolyte compartment 124 and a cathode 145 positioned or arranged in the catholyte compartment 144. The electrodes 125, 145 of the battery 10 can be electrically connected to one or more external electrical devices 170 such one or both of an electrical power source (e.g., wind, solar, or other (renewable) energy source to charge/recharge the battery 10) and an electrical load (e.g., any machine or other apparatus electrically driven by the battery 10, electrical power grid, etc.). The anolyte reservoir 122 can be in fluid communication with the anolyte compartment 124 to circulate anolyte solution 126 (e.g., via an anolyte pump 128) through the anolyte compartment 124 (e.g., contacting the anode and separator therein) and the anolyte reservoir 122. The catholyte reservoir 142 can be in fluid communication with the catholyte compartment 144 (e.g., via a catholyte pump 148) to circulate catholyte solution 146 through the catholyte compartment 144 (e.g., contacting the cathode and separator therein) and the catholyte reservoir 142.

The anolyte solution and catholyte solution can each contain an electrolyte. The electrolyte can be the same in each anolyte and catholyte solution. The electrolyte can contain the quinoxaline derivative according to Formula I. The quinoxaline derivative can be present in at least one of the anolyte solution and the catholyte solution. For example, the quinoxaline derivative can be present in one of the anolyte and catholyte solutions in an asymmetric cell configuration. For example, the quinoxaline derivative can be present in both anolyte and catholyte solutions in a symmetric cell configuration. In embodiments, the electrolyte can further include a positive electrolyte or charge carrier (e.g., in one or both the anolyte and/or catholyte for a symmetric or an asymmetric configuration), for example ferrocyanide (for neutral to alkaline conditions), permanganate (strongly alkaline conditions), ferrocene derivatives (neutral pH), bromide and iodide (acidic conditions).

The electrolyte can further comprise an aqueous medium. For example, the electrolyte can be an aqueous hydroxide solution of an alkaline or alkaline earth metal (e.g., NaOH, KOH).

The electrolyte can contain the quinoxaline derivative in a concentration of about 0.01 M or 0.1 M to 4 M. For example, the quinoxaline derivative can be present in a concentration of at least and/or up to 0.01, 0.02, 0.05, 0.1, 0.2, 0.4, 0.5, 0.6, 0.7, 0.8, 0.9, 1, 1.2, 1.5, 2, 2.5, 3, 3.5, or 4 M and ranges therebetween. The pH value of the electrolyte can broadly span pH 0-14.5, such as a pH of 0, 1, 2, 3, 4, 5, 6, 7, 8, 9, 10, 11, 12, 13, 14, 14.5, and ranges therebetween in an as-provided electrolyte solution or during operation of the battery.

### EXAMPLES

The following examples illustrate the disclosed methods, compositions, and apparatus, but are not intended to limit the scope of any claims thereto.

The relationship between capacity fade in aqueous quinoxaline-based RFBs and molecular decay was investigated by combining flow cell cycling with a suite of chemical analysis techniques, including ultraviolet-visible (UV-vis) spectroscopy, nuclear magnetic resonance (NMR) spectroscopy, and mass spectrometry using 2,3-dimethylquinoxaline-6-carboxylic acid (DMeQUIC) as a model quinoxaline. The conditions and energetic barriers to tautomerization of the reduced form of quinoxalines, Michael addition, dimerization, or hydrogenation were investigated by subjecting spectroscopic data obtained from operating flow cells to a Bayesian statistical inference protocol. Density Functional Theory (DFT) calculations were used to rationalize trends in the tautomerization energetics of quinoxaline derivatives and to find molecular design rules that can be used to identify tautomerization-resistant quinoxalines such as quinoxaline-2-carboxylic acid (2QUIC) that according to these examples did not exhibit capacity fade for about 220 h of flow cell cycling in a mixed symmetric configuration. In the following examples, Example 1 describes the decomposition mechanism and testing of comparative compound (DMeQUIC), Example 2 describes DFT calculations and results of tautomerization energies and relation to functional groups, and Example 3 describes experimental test results of selected compounds from Example 2.

### Materials and Methods

Chemicals. Sodium ferrocyanide (>98%), sodium hydroxide (>99%), and sodium chloride (>99%) were purchased from Sigma-Aldrich. 2,3-Dimethyl-quinoxaline-6-carboxylic acid (>97%), quinoxa-line-6-carboxylic acid (>95%), 2,3-dimethylquinoxaline (>97%), quinoxaline-2-carboxylic acid (>97%), 2,3-bis(bromomethyl)-quinoxaline (>97%), 3-amino-quinoxaline-2-carboxylic acid (QUI2C3A), sodium sulfite (98%), 3,4-diaminobenzoic acid (>97%), glyoxal sodium bisulfite (98%), dimethyl sulfoxide, and ethanol (absolute) were purchased from Fisher Scientific and used as received. Methanesulfonic acid-deuterium and methanesulfonic acid-sodium salt were purchased from Sigma-Aldrich and used as received. 2,3-Dimethyl-1,2,3,4-tetrahydro-quinoxaline-6-carboxylic acid was used as received from 1 ClickChemistry. All electrolyte solutions were prepared with deionized water (18 MΩ·cm).

Cyclic Voltammetry. Three-electrode cyclic voltammetry (CV) was conducted in 3 M NaOH, unless specified otherwise, using a 5 mm diameter glassy carbon disk electrode (Basi Inc.) along with a Ag/AgCl reference electrode (BaSI Inc.) and Pt wire (Basi Inc.) as the counter electrode at a scan rate of 50 mV/s. All CV measurements were conducted at a nominal active material concentration of 1 mM using a CHI7013E potentiostat (CH Instruments). The CVs were performed with an 85% resistance compensation.

Flow Cell Preparation and Cycling. Flow cells were assembled in a zero-gap configuration, as known in the art. Pyrosealed POCO graphite flow plates with serpentine flow patterns were used for both electrodes, and a 50 μm-thick NAFION 212 (Fuel Cell Store) was used as the membrane. Each electrode comprised a 5 cm² sheet of CE Tech GF020 graphite felt (Fuel Cell Store, 2.1 mm thick). The electrodes were oven-dried in air for 12 h at 400° C. prior to use, whereas membranes were pre-soaked in the supporting electrolyte for 24 h before use in the cell. A flow rate of 50 mL/min was used in the cell cycling and operando flow cell experiments. The electrolytes were degassed with nitrogen prior to cycling and the UV-vis measurements were performed in the presence of nitrogen. Sodium ferrocyanide (Na₄Fe(CN)₆) was used in the non-capacity-limiting electrolyte in all of the compositionally asymmetric cells. Unless otherwise noted, cycling experiments were performed using a constant-current, constant-voltage cycling protocol to access the entire capacity of the capacity-limiting electrolyte. In this protocol, a constant current is applied to the cell until a specified potential limit is reached; the cell is then held at that potential until the current density reaches an absolute value of 1 mA/cm². A Biologic VSP potentiostat was used for cell cycling measurements.

Permeability Measurements. Membrane permeability was measured using a lab-made glass H-cell with a liquid volume of 6 mL on each side arm along with Viton gaskets to hold the membrane with an opening of 0.9 cm². The donating side was filled with the charge carrier and supporting electrolyte, whereas the receiving side contained the supporting electrolyte only. The concentration of crossed-over material in the receiving side was characterized using UV-vis spectrophotometry, and the permeability was calculated from the slope of crossed over concentration versus time based on Fick's law.

Ultraviolet-Visible Spectroscopy. UV-vis spectroscopy was conducted on an Ocean-HDX-UV-VIS spectrometer HDX00518 (Ocean Insight). Spectroscopic measurements were taken using 50 and 25 msec integration times for the ex-situ and operando experiments, respectively, and averaged over 50 scans in both cases. The operando UV-vis crossflow cell (Firebird Optics) had a path length of 60 μm. Unless otherwise noted, all ex-situ measurements were conducted at a nominal 0.1 mM concentration of the active material in a supporting electrolyte of 3 M NaOH.

Mass Spectrometry and NMR Spectroscopy. Mass spectrometry (MS) was performed using an Agilent 6230 TOF Mass spectrometer with a C18 column on samples diluted to 100 μM in water with an injection volume of 20 μL. All measurements were carried out in negative ion mode. All ¹H and ¹³C NMR spectra were acquired at room temperature in deuterated solvents using a Bruker Avance Neo 500. ¹H NMR spectra were taken using a 2 s relaxation delay, and ¹³C NMR spectra had a 1 s relaxation delay. All chemical shifts were reported in units of δ (ppm) relative to tetramethylsilane (TMS) and referenced to residual solvent. Peak multiplicities were reported in the following manner: singlet(s), doublet (d), doublet of doublets (dd), triplet (t), quartet (q), multiplet (m), and broad signal (br). A 0.1 M NaOD solution and a 0.05 M CH₃SO₃Na or CH₃SO₃D (as specified for each experiment) in D₂O were used as internal standard. 150 μL of the sample solution were mixed with 600 UL of the internal standard, and 600 μL of the resulting solution were entered into the NMR tube.

### Example 1

This example describes the characterization of the charge-discharge behavior of DMeQUIC as a model quinoxaline and the mechanistic analysis of the decomposition mechanism of the model compound. Kinetic rate constants for tautomerization were determined via experimental characterization of the model compound, which in turn demonstrated that tautomerization of the reduced form of quinoxalines is primarily responsible for capacity fade in a redox flow battery.

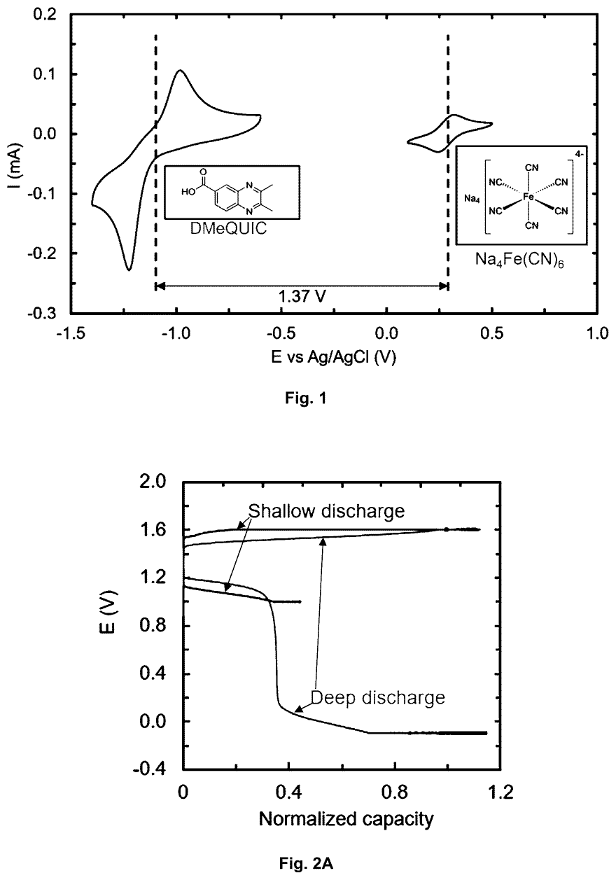

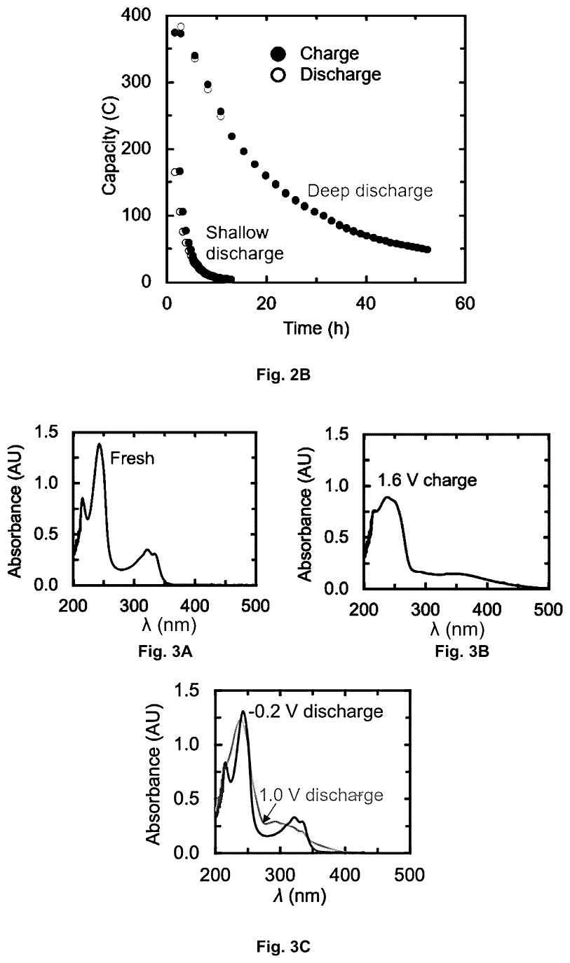

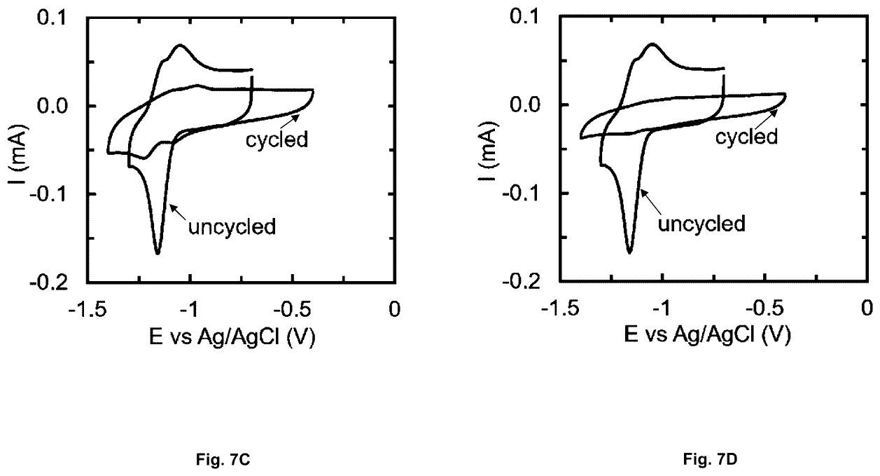

DMeQUIC was used as a model quinoxaline due to its commercial availability, low reduction potential (−1.09 vs Ag/AgCl at pH 13) and its moderately fast redox kinetics (260 mV separation between cathodic and two anodic redox peaks at a scan rate of 50 mV/s). The low reduction potential of DMeQUIC was linked to high open-circuit potentials against common positive electrolyte charge carriers: 1.37 V against ferrocyanide (FIG. 1) and 1.55 V against permanganate. The charge-discharge mechanism of the model molecule was studied by cycling an electrolyte containing 0.4 M DMeQUIC against sodium ferrocyanide in an alkaline (pH 13) flow cell at 20 mA/cm² using a constant current-constant voltage (CCCV) protocol. It was found that the current efficiency and rate of capacity fade were strongly dependent on the potential limit applied on discharge (FIG. 2A-2B). During the first cycle (FIG. 2A), two voltage plateaus were present upon discharge, centered at about 1.1 and 0.0 V. About 40% of the charging (DMeQUIC reduction) capacity was recovered at the higher plateau, and the remaining capacity was recovered at or below the lower plateau. This suggested that the reduced form of DMeQUIC decomposed into at least one product that could be converted back to DMeQUIC at a high oxidative overpotential. These potential-dependent variations in current efficiency manifested as differences in capacity retention during long-term CCCV cycling (FIG. 2B) cycling at a discharge potential limit of 1.0 V (shallow discharge) yielded a 90% loss of capacity within 5 h, whereas a similar capacity loss took about 60 h for cycling at a limit of −0.1 V (deep discharge). UV-vis (FIG. 7A-7B) and voltametric (FIG. 7C-7D) analysis of the cycled DMeQUIC electrolytes revealed a clear change in chemical composition and decrease in the redox activity, respectively, indicating that the capacity fade observed in FIG. 2B originated from the decomposition of and loss of redox activity in the DMeQUIC electrolyte.

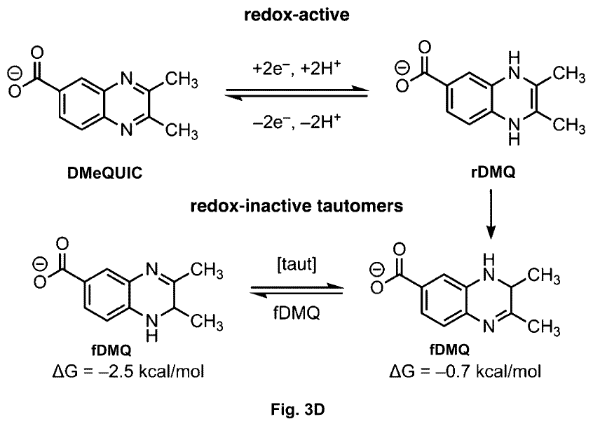

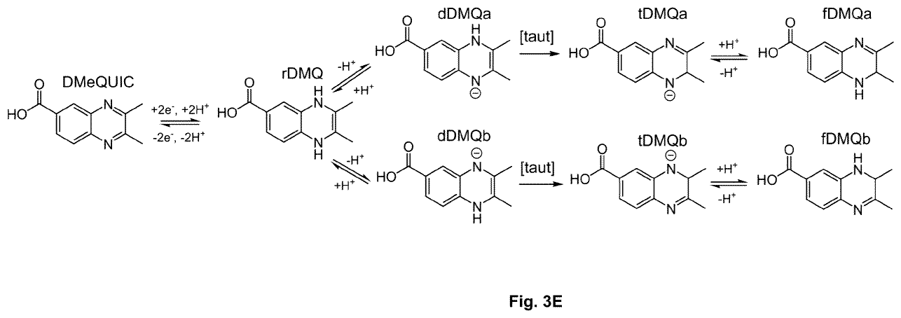

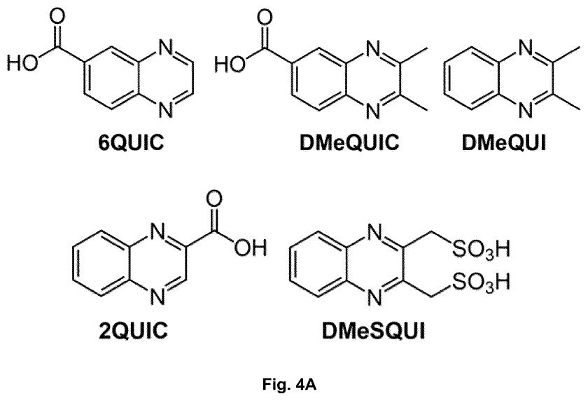

The decomposition of the reduced form of DMeQUIC and its recovery upon deep discharge were supported by operando UV-vis (FIG. 3A-3C) and ex situ ¹H NMR spectroscopic analyses. Reduction (during charging) of DMeQUIC resulted in distinct shifts in its UV-vis absorbance (FIG. 3B) and ¹H NMR spectra. The NMR spectrum of the charged electrolyte suggested at least two components, consistent with reduced DMeQUIC and at least one decomposition product. DMeQUIC displayed two singlet peaks at ca. 2.1 ppm, originating from the pair of methyl groups at the 2 and 3 positions of the pyrazine core, which each integrate to 3H. Three peaks were identified in the aromatic region of the spectrum, one at 7.4 ppm (doublet), one at 7.0 (singlet), and one at 7.6 ppm (doublet), integrating to three protons, as expected for this substitution pattern. These peaks should have been retained, but shifted, in the reduced (charged) form, with the addition of two N—H protons, which may not have shown due to exchange with the deuterated NMR solvent. Instead, the methyl groups became more differentiated, with one appearing at 2.0 ppm (similar to DMeQUIC) and another more up field at 1.0 ppm, indicating that the latter was in a new chemical environment. In addition, there were new peaks at 3.8 ppm, which were believed to correspond to new protons at the 2 and 3 positions on the pyrazine core (which were not present in reduced DMeQUIC). The aromatic region showed five peaks, providing further evidence for two or more degradation products. The original UV-vis and NMR spectra for DMeQUIC were recovered after discharge at −0.2 V but not at 1.0V (FIG. 3C).

Bayesian Inference has been shown in the art to be effective in the analysis of UV-vis data from operating flow cells to identify the individual spectra of decomposition products and their respective rates of evolution, thus quantitatively connecting charge carrier decomposition to capacity fade. These methods were used here to calculate the rate constants of decomposition of comparative model molecule DMeQUIC from a redox active compound into redox inactive species using the UV-vis spectra collected during cycling of an electrolyte containing DMeQUIC. As illustrated in FIG. 3D and FIG. 3E, upon reduction, DMeQUIC converts to rDMQ, which decomposes into a transient, redox-active intermediate, tDMQ, and later into a final, redox-inactive form, fDMQ according to the

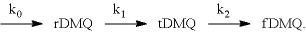

FIG. 3D also shows that conversion into redox-inactive forms via tautomerization is favored for DMeQUIC with negative free energies of tautomerization. All of the reaction steps were modeled with first-order kinetics. Calculations are described in further detail in Modak et. al. (J. Am. Chem Soc., 2024, vol. 146, pp. 5173-5185), which is incorporated herein by reference in its entirety.

UV-vis spectra of a 5 mM DMeQUIC electrolyte were collected during a 16 h potentiostatic reduction in a DMeQUIC-Fe(CN)₆ flow cell. DMeQUIC peaks at 245, 325, and 335 nm fell within the first 2 min of reduction, at the expense of a broad peak centered at 365 nm, which rose and grew until 10 min, before falling gradually. The diminution of the 365 nm peak coincided with the emergence of two new peaks at 235 and 293 nm. The Bayesian inference analysis provided that the observed time-varying absorbance profiles were explained by the successive evolution of three unique species. Given the cell cycling data, it was believed that the pure spectra represented DMeQUIC, its reduced form (rDMQ), a transient but redox-active decay product of the reduced form (tDMQ), and a final redox-inactive species (fDMQ). From the concentration profiles, first-order rate constants for the conversion of DMeQUIC to rDMQ, rDMQ to tDMQ, and tDMQ to fDMQ were calculated to be 2.0×10⁻², 9.5×10⁻⁵, and 1.4×10⁻⁵ s⁻¹, respectively. First-order kinetics was supported by similar rates of molecular conversion upon reduction of DMeQUIC at concentrations of 4, 3, and 1 mM. The concentration independence of these rates was inconsistent with DMeQUIC decay being rate-limited by a bimolecular mechanism such as dimerization.

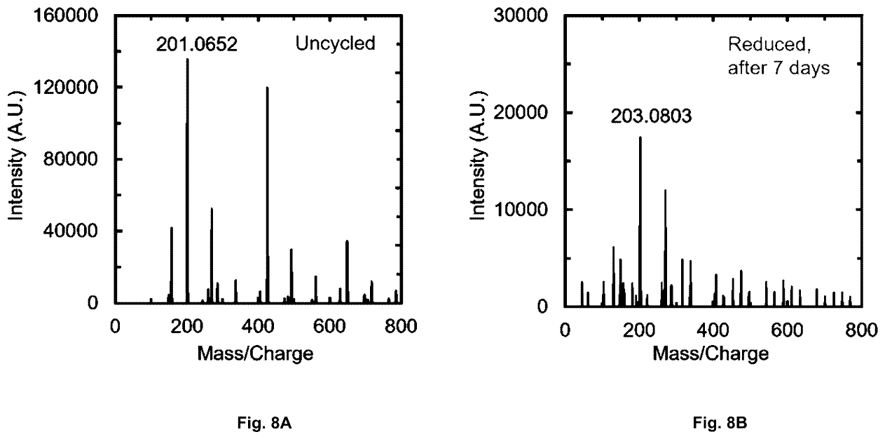

The spectroscopically derived rate constants were quantitatively consistent with the capacity fade rates in FIG. 2B. Assuming that capacity loss during shallow-discharge cycling was controlled by conversion of rDMQ to tDMQ yielded a rate constant of 1.8×10⁻⁴ s⁻¹, which was in good agreement with the corresponding value from spectroscopic/Bayesian analysis on the 5 mM DMeQUIC data. A similar estimation of the rate constant for conversion of tDMQ to fDMQ from cycling to the lower discharge potential of −0.1 V yielded 2.2×10⁻⁵ s⁻¹, which was again in good agreement with the analogous value from the Bayesian analysis of the spectra. Analysis of electrochemically reduced DMeQUIC solutions using NMR spectroscopy and liquid chromatography-mass spectrometry (LC-MS) revealed that tautomerization accounted for the observed decomposition. Tautomer formation was further supported by NMR analysis of fully reduced aged DMeQUIC solutions. LC-MS data (FIG. 8A-8B) provided additional evidence for tautomerization and against other potential decomposition mechanisms, such as Michael addition and full hydrogenation of the pyrazine core. To further rule out the fully hydrogenated species, the fully reduced quinoxaline standard was purchased and measured, this standard displayed distinct 1H and 13C NMR spectra compared to the observed decomposition product.

Without intending to be bound by any theory and although the exact structure of the intermediate tDMQ is not known, as illustrated in FIG. 3E, it is believed that tDMQ is the nitrogen-deprotonated form of fDMQ, and that under alkaline conditions imine-enamine tautomerization can proceed via a nitrogen-deprotonated enamine (dDMQ) and a nitrogen-deprotonated imine intermediate (tDMQ), and that tautomerization is the dominant decomposition mechanism of quinoxalines and more likely to occur over dimerization, Michael addition, or full hydrogenation.

### Example 2

This example is intended to show the effect of quinoxaline functionalization on reduced quinoxaline tautomerization energetics and quinoxaline stability.

Density Functional Theory (DFT) modeling. DFT calculations were used to predict energies for tautomerization, Michael attack, and dimerization, as well as redox potentials and pKₐ values of various quinoxaline derivatives. All DFT calculations were performed using the NWChem software with the B3LYP exchange-correlation functional and the Def2-TZVPPD basis set. The COSMO implicit solvation model with default parameters was used during geometry optimization to implicitly treat molecule solvation by water. A self-consistent field convergence energy of 1×10⁻⁶ au was used. To maintain charge neutrality, the carboxylic acid and sulfonic acid functional groups were modeled without deprotonation.

The following equations were used to calculate the Gibbs free energies of reduction (ΔG_{red}) and tautomerization (ΔGₜₐᵤₜ): ΔG_{red}=Gₑₙₐₘᵢₙₑ−G_{H}_{₂}−G_{oxidized} and ΔGₜₐᵤₜ=Gᵢₘᵢₙₑ−Gₑₙₐₘᵢₙₑ, where Gₑₙₐₘᵢₙₑ, Gᵢₘᵢₙₑ, and G_{oxidized} are the DFT-calculated Gibbs free energies of the enamine form (i), imine form (iv), and oxidized form of the given quinoxaline according to the model reaction:

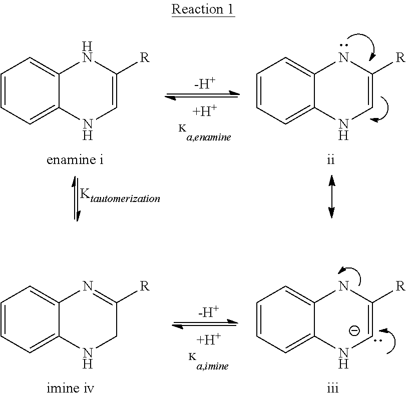

Additional details regarding the DFT calculations are described in Modak et. al. (J. Am. Chem Soc., 2024, 146, 5173-5185), which is incorporated herein by reference in its entirety.

Three molecular properties that influence the tautomerization energetics and its link to molecular structure were studied: (1) redox potential, (2) kinetic parameters and relative pKₐ values of the enamine and imine forms and their connection to the strength of the electron-withdrawing group (EWG), and (3) hydrogen bonding between the pyrazine N—H and an H-acceptor functional group. A total of 18 molecules (FIG. 4A-4B) were computationally modeled, including quinoxaline-2,3-disulfonic acid-6-carboxylic acid (DS6QUIC), quinoxaline (QUI), 2-acetylquinoxaline (2COCH3QUI), quinoxaline-2,3-dicarbonitrile (DCNQUI), quinoxaline-5-car-boxylic acid (5QUIC), quinoxaline-5,8-dicarboxylic acid (5,8-QUIDC), quinoxaline-2,3-dicarbonitrile-6,7-disulfonic acid (DCNQUI-6,7-DS), quinoxaline-2-carbonitrile (2CNQUI), 2-nitroquinoxaline (2NO2QUI), quinoxaline-2-amine (2NQUI), 3-amino-quinoxaline-2-carboxylic acid (QUI2C3A), 2-methylquinoxaline (2MeQUI), and quinoxa-line-2-carbaldehyde (2CHOQUI). FIG. 4C shows the DFT-computed redox potentials at pH 13 against the free energy of tautomerization for all 18 quinoxalines. A weak but positive correlation between the redox potential and tautomerization energy was identified (R²=0.49). Without intending to be bound by theory, it is believed that there is a trade-off between a high open-circuit potential and resistance to tautomerization.

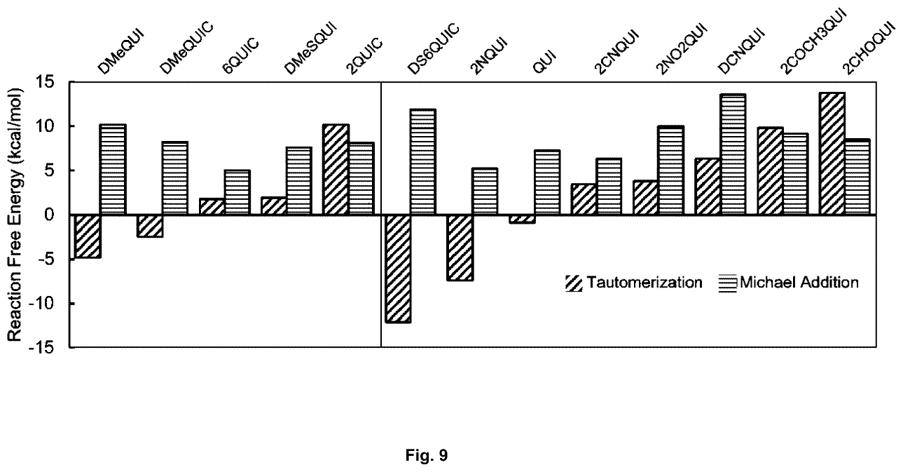

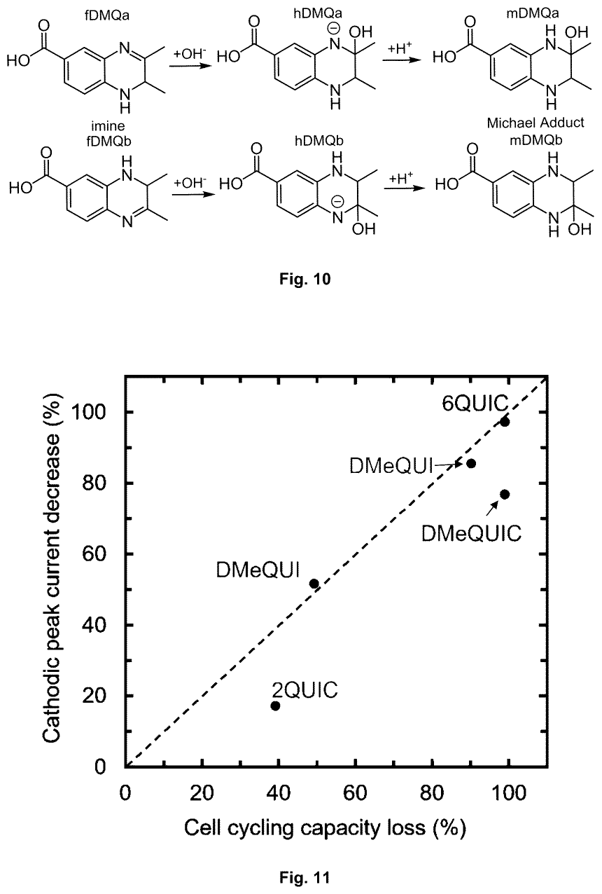

The DFT-calculated redox potentials of experimentally studied quinoxalines (indicated by squares in FIG. 4C), which fell between −0.8 and −1.2 V versus Ag/AgCl at pH 13, agreed moderately well with experimentally determined values (Table 1 below). Five of the 18 molecules were experimentally tested (Example 3), for those molecules, tautomerization was predicted to be exothermic for the reduced forms of DMeQUI and DMeQUIC, whereas it was slightly endothermic for 6QUIC and DMeSQUI (~2 kcal/mol) and highly endothermic (~10 kcal/mol) for 2QUIC (FIG. 9). Generally, Michael addition (FIG. 10) was predicted to be endothermic and was typically less thermodynamically favorable compared with tautomerization (FIG. 9), consistent with experimental observations. Likewise, dimerization of the quinoxaline derivatives was computed to be generally very endothermic (e.g., 55.9, 52.1, 53.6, and 48.4 kcal/mol for the reduced forms of 2QUIC, 6QUIC, DMeQUIC, and DMeQUI, respectively). Intuitively, the redox potential and tautomerization energy are related to the strength of the N—H bond of the pyrazine ring, which was modulated by the functional groups. A stronger N—H bond made tautomerization from the enamine to the imine form more unfavorable because this process required breaking the N—H bond and forming a C—H bond. In addition, it made reduction, which involves the formation of the N—H bond, more favorable. This analysis suggested that a strong N—H bond led to favorable reduction, a more positive redox potential, and less favorable tautomerization.

Analysis of the reaction kinetics (Hammett constants) revealed a relationship between the EWG strength of the quinoxaline substituents and the tautomerization energetics. Quinoxaline derivatives with stronger EWGs presented a more stable enamine form relative to the imine form, making tautomerization less favorable. The Hammett plot in FIG. 4D shows the effect of the σ-Hammett constant of the substituent adjacent to the pyrazine N (substituent R in Reaction 1) and the tautomerization free energy when there are no substituents in any other positions (that is, when only substituent R is changed). This plot showed a positive correlation between σ⁻ and tautomerization energy (R²=0.66). Without intending to be bound by theory, it is believed that a stronger EWG in the position adjacent to pyrazine N can result in a greater resistance to tautomerization.

Another factor that results in unfavorable tautomerization is the stabilization of the enamine structure by forming a hydrogen bond between the H on the pyrazine N and a hydrogen bond acceptor group in an adjacent substituent, which may have contributed to the stability of 2QUIC to tautomerization. Also, because only one of the two pyrazine N—H groups of 5QUIC is hydrogen-bonded to the carboxylic acid group, only one of the two possible tautomerization pathways required breaking the hydrogen bond. This pathway was associated with an unfavorable free energy change (+8.6 kcal/mol), in contrast to a slightly favorable free energy change associated with the alternative pathway that did not include breaking of the hydrogen bond (−1.0 kcal/mol). This observation suggested that the formation of a hydrogen bond with both pyrazine H atoms would enable strong resistance to tautomerization, and indeed, 5,8-QUIDC has a DFT-calculated tautomerization free energy of +11.5 kcal/mol, more unfavorable than that for 5QUIC. In theory, the two carboxylic acid groups of 5,8-QUIDC make this molecule not only resistant to tautomerization but also highly soluble in water, making it a promising derivative for resisting capacity fade in aqueous organic redox flow batteries.

Without intending to be bound by theory it is believed that electron-withdrawing group strength and H-bonding strength can be used to identify tautomerization resistant quinoxalines.

### Example 3

This example presents the results of flow cell cycling of quinoxaline derivatives carried out to evaluate the influence of functionalization on the cycling stability of a subset of the quinoxaline derivatives analyzed by DFT.

The molecules tested are shown in FIG. 4A and include DMeQUIC, DMeQUI, 6QUIC, and 2QUIC which were procured commercially, whereas DMeSQUI was synthesized for this study via a simple, one-step reaction as follows: 2,3-bis(bromomethyl)-quinoxaline (0.632 g, 2 mmol) was reacted with dimethyl sulfoxide (DMSO, 6 mL) and aqueous 1.1 M Na2SO3 (4 mL) at 100° C. for 16 h in a sealed vial. The resulting solution was cooled to room temperature and filtered; the filtrate reduced to 5 mL by evaporation and mixed with ethanol (50 mL). The resulting suspension was filtered and the precipitate dried and tested by 1H NMR to corroborate the identity of the compound. The other derivatives analyzed by DFT that have endothermic tautomerization energies were not commercially available, with the exception of QUI2C3A. However, the CV of QUI2C3A displayed a 1.1 V separation between cathodic and anodic redox peaks, which renders cycling of this molecule in a flow battery impractical.

First-order capacity fade rate constants were estimated from the slope of the linear fit of normalized discharge capacity on a logarithmic scale against time and were found to vary over 3 orders of magnitude (Table 1), increasing in the following order: 2QUIC<DMeSQUI<DMeQUI<DMeQUIC<6QUIC. CV measurements were conducted on all cycled electrolytes to experimentally determine the redox potential (Table 1), and a strong correlation between the decrease in peak cathodic current and capacity lost during flow cell cycling was observed (FIG. 11). Table 1 also includes for comparison the DFT-calculated redox potential values (Example 2) for the five experimentally tested compounds

TABLE 1

| | Fade rate | Redox potential (V vs Ag/AgCl) | |
|---|---|---|---|
| Molecule | constant (s⁻¹) | Experimental | DFT Model |
| 2QUIC | 5.31 ± 0.04 × 10⁻⁷ | −0.95 | −0.83 |
| DMeSQUI | 4.33 ± 0.05 × 10⁻⁶ | −1.01 | −1.02 |
| DMeQUI | 2.71 ± 0.07 × 10⁻⁵ | −1.10 | −1.17 |
| DMeQUIC | 8.9 ± 0.2 × 10⁻⁵ | −1.12 | −1.05 |
| 6QUIC | 3.5 ± 0.1 × 10⁻⁴ | −1.00 | −0.93 |

DFT-predicted tautomerization energetics rationalized experiments for quinoxaline stability; for example, the most stable derivative tested, 2QUIC, was predicted to have very endothermic tautomerization (ΔGₜₐᵤₜ=+10.2 kcal/mol), whereas the less stable molecules such as DMeQUIC and 6QUIC had values of ΔGₜₐᵤₜ of −2.5 and 1.8 kcal/mol, respectively. Quantitative correlations between tautomerization energetics to measured decay rates were not made since the predicted thermodynamic driving force does not directly consider kinetic barriers, which can play a role in driving tautomerization. Nevertheless, it was noted that the 4.6%/day capacity fade rate for the 2QUIC cell was about 2 orders of magnitude slower than that of DMeQUIC. Without intending to be bound by theory, it is believed that functionalization can play an important role in stabilizing quinoxalines against tautomerization upon reduction.

Because the permeability of NAFION to organic charge carriers under aqueous conditions translates to a capacity fade rate constant of up to about 6×10⁻⁷ s⁻¹, it was evaluated the possibility that molecular crossover rather than decomposition accounted for most of the apparent capacity fade in the 2QUIC cell. The permeability of NAFION 212 to 2QUIC was measured using an H-cell to be 2.5×10⁻⁹ cm²/s. Based on this permeability, capacity fade from crossover was calculated to be about 6.9%/day, which was close to the 4.6%/day fade rate measured in the 2QUIC cell.

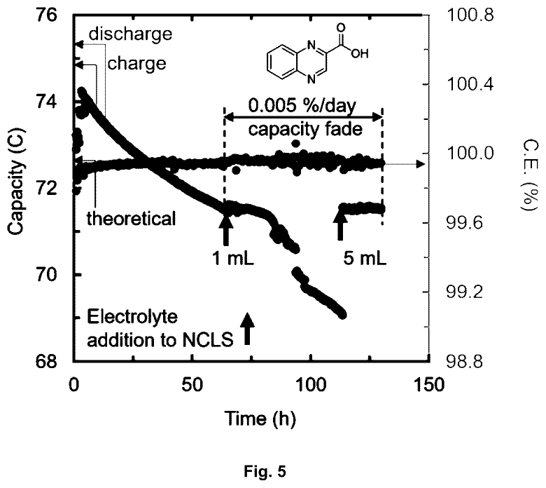

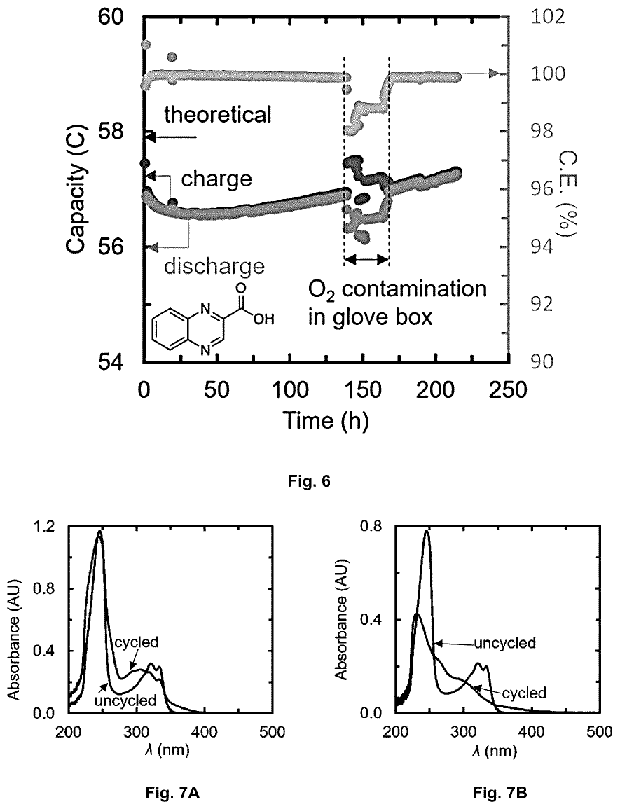

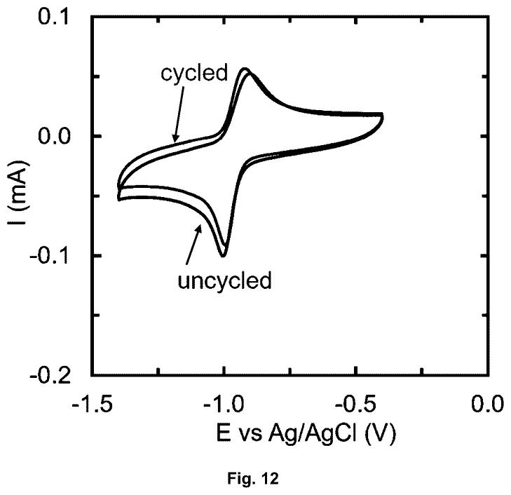

Further, to assess the intrinsic chemical stability of 2QUIC, a volumetrically unbalanced, compositionally symmetric cell was cycled. The cell contained 0.05 M 2QUIC and exhibited a capacity fade rate of 0.005%/day over 65 h of CCCV cycling (FIG. 5). 2QUIC in addition has a low equivalent weight and a negative redox potential, which may lower costs at the system level. A mixed symmetric cell was cycled that was capacity-limited by 2QUIC but had 2QUIC and ferrocyanide on both sides to limit crossover-induced capacity fade. The cell had an open-circuit potential of 1.2 V and did not exhibit capacity fade (FIG. 6) over 220 h of CCCV cycling between 0.8 and 1.4 V. It rather showed a small increase in capacity that cumulatively amounted to <1% of the initial redox capacity of 2QUIC. The increase is equivalent to a gain in volume of about 30 μL and might have been caused by slow entrainment of initially stranded electrolyte into the main flow circuit. CVs of 2QUIC in the cycled and uncycled electrolytes from the capacity-limiting side of the cell were virtually identical (FIG. 12), indicating negligible change in the redox capacity of the electrolyte. Although low 2QUIC concentrations (≤0.1 M) were used in the foregoing cycling measurements, it is likely that 2QUIC could be cycled at higher concentrations. Accordingly, the solubility of 2QUIC in 1 M NaOH and 1 M KOH solutions were measured as 0.45 M and 1.22 M respectively, the latter of which translates to a high volumetric capacity of 65 A h/L.

Modak et. al. (“Substituent Impact on Quinoxaline Performance and Degradation in Redox Flow Batteries,” J. Am. Chem Soc., 2024, vol. 146, pp. 5173-5185) and Modak (“Enabling Low-Cost Electrolytes and Membranes for Redox Flow Batteries,” University of Michigan Ph.D. dissertation (publicly available Feb. 13, 2024)) provide additional disclosure, computational methods and results, and experimental results, each of which is incorporated herein by reference in its entirety.

Because other modifications and changes varied to fit particular operating requirements and environments will be apparent to those skilled in the art, the disclosure is not considered limited to the examples chosen for purposes of illustration and covers all changes and modifications which do not constitute departures from the true spirit and scope of this disclosure.

What is claimed is:

1. A redox flow battery comprising:
an electrochemical cell comprising an electrolyte comprising a quinoxaline derivative according to Formula I: 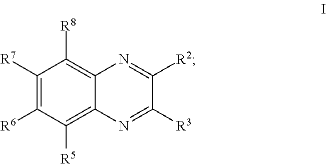
wherein:
R², R³, R⁵, R⁶, R⁷, and R⁸ are each independently selected from the group consisting of H, a hydrocarbon group containing 1 to 10 carbon atoms, a nitro group, a cyano group, a carbonyl group, a sulfonate group, a sulfonyl group, a hydroxyl group, an ether group, an amino group, a thio group, a phosphino group, and any of the foregoing linked to the quinoxaline ring via a hydrocarbon linking group containing 1 to 10 carbon atoms; and
at least one of conditions (A) and (B) is satisfied:
(A) R² is selected from the group consisting of a carbonyl group, a cyano group, or a nitro group; and
(B) R⁵ and R⁸ are each independently selected from the group consisting of a carbonyl group, a cyano group, or a nitro group.

2. The redox flow battery of claim 1, wherein R² is a carbonyl group.

3. The redox flow battery of claim 1, wherein:
R² is selected from the group consisting of —C(═O)OH, —C(═O)H, and —C(═O)CH₃; and
R³, R⁵, R⁶, R⁷, and R⁸ are each H.

4. The redox flow battery of claim 1, wherein R⁵ and R⁸ are each carbonyl groups.

5. The redox flow battery of claim 4, wherein:
R⁵ and R⁸ are each —C(═O)H; and
R², R³, R⁶, and R⁷ are each H.

6. The redox flow battery of claim 1, wherein R² is a cyano group.

7. The redox flow battery of claim 6, wherein:
(a) R² is —CN; and R³, R⁵, R⁶, R⁷, and R⁸ are each H;
(b) R² and R³ are each —CN; and R⁵, R⁶, R⁷, and R⁸ are each H; or
(c) R² and R³ are each —CN; R⁵ and R⁸ are each H; and R⁶ and R⁷ are each —SO₃H.

8. The redox flow battery of claim 1, wherein R² is a nitro group.

9. The redox flow battery of claim 8, wherein:
R² is —NO₂; and
R³, R⁵, R⁶, R⁷, and R⁸ are each H.

10. The redox flow battery of claim 1, wherein the quinoxaline derivative according to Formula I has a separation between the anodic and cathodic redox potential lower than 0.5 V as measured by cyclic voltammetry at a scan rate of 50 mV/s in an alkaline medium.

11. The redox flow battery of claim 1, wherein the quinoxaline derivative according to Formula I has a fade rate constant of about 1×10⁻⁶/s or less.

12. The redox flow battery of claim 1, wherein the redox flow battery (or electrochemical cell thereof) exhibits a molecular loss rate of about 0.1%/day or less.

13. The redox flow battery of claim 1, wherein the quinoxaline derivative according to Formula I has a tautomerization free energy (ΔGₜₐᵤₜ) greater than 0 kcal/mol.

14. The redox flow battery of claim 1, wherein the quinoxaline derivative according to Formula I has a redox potential in a range of −0.6 V to −1.8 V.

15. The redox flow battery of claim 1, wherein the quinoxaline derivative according to Formula I has an equivalent weight of 300 g/mol e stored or less.

16. The redox flow battery of claim 1, wherein the quinoxaline derivative according to Formula I has a solubility of at least 0.05 M in the electrolyte.

17. The redox flow battery of claim 1, further comprising:
an anolyte reservoir in fluid communication with the electrochemical cell;
a catholyte reservoir in fluid communication with the electrochemical cell;
a separator dividing the electrochemical cell into an anolyte compartment and a catholyte compartment;
an anode arranged in the anolyte compartment; and
a cathode arranged in the catholyte compartment;
wherein:
the anolyte reservoir is in fluid communication with the anolyte compartment to circulate anolyte solution through the anolyte compartment and the anolyte reservoir;
the catholyte reservoir is in fluid communication with the catholyte compartment to circulate catholyte solution through the catholyte compartment and the catholyte reservoir; and
the electrolyte comprising the quinoxaline derivative according to Formula I is present in at least one of the anolyte solution and the catholyte solution.

18. The redox flow battery of claim 1, wherein the electrolyte further comprises an aqueous medium with the quinoxaline derivative according to Formula I in solution in the aqueous medium.

19. A redox flow battery comprising:
an electrochemical cell comprising an electrolyte comprising a substituted quinoxaline, wherein the substituted quinoxaline comprises at least one of (A) a carbonyl group, a cyano group, or a nitro group at position 2, and (B) a carbonyl group, a cyano group, or a nitro group at positions 5 and 8.

20. A redox flow battery comprising:
an electrochemical cell comprising an electrolyte comprising a quinoxaline derivative according to Formula I: 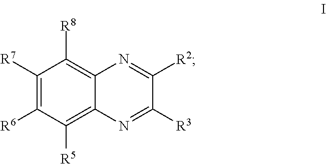
wherein:
R², R³, R⁵, R⁶, R⁷, and R⁸ are each independently selected from the group consisting of H, a hydrocarbon group containing 1 to 10 carbon atoms, a nitro group, a cyano group, a carbonyl group, a sulfonate group, a sulfonyl group, a hydroxyl group, an ether group, an amino group, a thio group, a phosphino group, and any of the foregoing linked to the quinoxaline ring via a hydrocarbon linking group containing 1 to 10 carbon atoms; and
at least one of conditions (A)-(E) is satisfied:
(A) the quinoxaline derivative according to Formula I has a separation between the anodic and cathodic redox potential lower than 0.5 V as measured by cyclic voltammetry at a scan rate of 50 mV/s in an alkaline medium;
(B) the quinoxaline derivative according to Formula I has a fade rate constant of 1×10⁻⁶/s or less;
(C) the redox flow battery (or electrochemical cell thereof) exhibits a molecular loss rate of 0.1%/day or less;
(D) the quinoxaline derivative according to Formula I has a tautomerization free energy (ΔGₜₐᵤₜ) greater than 0 kcal/mol; and
(E) the quinoxaline derivative according to Formula I has a redox potential in a range of −0.6 V to −1.8 V.

21. The redox flow battery of claim 20, wherein all of conditions (A)-(E) are satisfied.

22. A quinoxaline derivative according to Formula I: 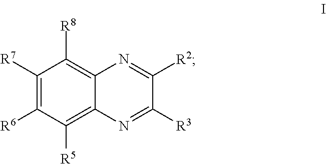
wherein:
R², R³, R⁵, R⁶, R⁷, and R⁸ are each independently selected from the group consisting of H, a hydrocarbon group containing 1 to 10 carbon atoms, a nitro group, a cyano group, a carbonyl group, a sulfonate group, a sulfonyl group, a hydroxyl group, an ether group, an amino group, a thio group, a phosphino group, and any of the foregoing linked to the quinoxaline ring via a hydrocarbon linking group containing 1 to 10 carbon atoms; and
optionally, at least one of conditions (A)-(D) is satisfied:
(A) the quinoxaline derivative according to Formula I has a separation between the anodic and cathodic redox potential lower than 0.5 V as measured by cyclic voltammetry at a scan rate of 50 mV/s in an alkaline medium;
(B) the quinoxaline derivative according to Formula I has a fade rate constant of 1×10⁻⁶/s or less;
(C) the quinoxaline derivative according to Formula I has a tautomerization free energy (ΔGₜₐᵤₜ) greater than 0 kcal/mol; and
(D) the quinoxaline derivative according to Formula I has a redox potential in a range of −0.6 V to −1.8 V.

23. An electrolyte solution comprising:
an aqueous medium; and
a quinoxaline derivative according to claim 22 in solution in the aqueous medium.

24. The electrolyte solution of claim 23, wherein the quinoxaline derivative is present in a concentration of at least about 0.01 M.

25. An electrical power system comprising:
the redox flow battery of claim 1; and
at least one of an electrical power source and an electrical load in electrical connection with the redox flow battery.
